(19) United States
(12) Patent Application Publication
(10) Pub. No.: US 2010/0236569 A1
(43) Pub. Date: Sep. 23, 2010

(54) COSMETIC COMPOSITION COMPRISING A BRANCHED SULPHONIC POLYESTER AND A THICKENER AND USES IN HAIR STYLING

(76) Inventors: Joathan GAWTREY, Boulogne (FR); Cécile Bebot, Clichy (FR); Dorothée Pasquet, Bois Colombes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/640,591

(22) Filed: Dec. 17, 2009

Related U.S. Application Data
(60) Provisional application No. 61/142,243, filed on Jan. 2, 2009.
(60) Provisional application No. 61/146,783, filed on Jan. 23, 2009.

(30) Foreign Application Priority Data
Dec. 17, 2008 (FR) 0858672
Dec. 17, 2008 (FR) 0858678

(51) Int. Cl. A45D 7/06; A61K 8/85; A61Q 5/06

(52) U.S. Cl. 132/206; 424/70.13; 424/70.9

(57) ABSTRACT

The present application relates to a cosmetic composition comprising, in a cosmetically acceptable medium: at least one fixing branched sulphonic polyester and at least one polysaccharide thickener comprising at least one glucose unit. The present application also relates to the uses of this composition for example for the styling and the shaping of the hair.

This application claims benefit of U.S. Provisional Application No. 61/142,243, filed Jan. 2, 2009, and U.S. Provisional Application No. 61/146,783, filed Jan. 23, 2009. This application also claims benefit of priority under 35 U.S.C. §119 to French Patent Application Nos. 0858672 and 0858678, filed Dec. 17, 2008.

The present disclosure relates to novel cosmetic compositions comprising at least one fixing branched sulphonic polyester and at least one polysaccharide thickener comprising at least one glucose unit, and also to the uses of these compositions, for example, in styling.

The present disclosure also relates to a method of styling keratin materials using these compositions.

The use of branched sulphonic polyesters in hair styling and fixing compositions is known and described, for example, in patent applications EP 0 966 946, WO 98/38969, and WO 99/63955.

Nevertheless, the use of branched sulphonic polyesters is not without drawbacks:

the use of these polyesters in hair lacquers containing large quantities of alcohol generally gives good styling properties but does not necessarily make it possible to obtain sufficient lacquering power;

the application of these polyesters in the form of lacquers with a high alcohol content can give the hair, after brushing, a dry feel; this undesirable phenomenon is for example noticeable for dyed hair, and

these polyesters are generally in semi-solid form, and this property often makes their use difficult, for example, to ensure a homogeneous distribution of these polyesters over all the hair to be treated.

Applicant has demonstrated that the use of at least one fixing branched sulphonic polyester with at least one polysaccharide thickener comprising at least one glucose unit makes it possible to overcome at least one, and in certain embodiments all of the aforementioned drawbacks.

One subject of the present disclosure is a cosmetic composition comprising, in a cosmetically acceptable medium:

at least one fixing branched sulphonic polyester; and

at least one polysaccharide thickener comprising at least one glucose unit.

The compositions obtained may be in the form of gels, mousses, sprays, creams, or pastes.

The compositions according to the present disclosure can be easy to prepare and to apply. They remain satisfactorily localized, without runs, at the point of application. The compositions according to the present disclosure may be applied accurately, without runs and without a reduction in viscosity over time.

Moreover, the compositions according to the present disclosure make it possible to give the hairstyle a natural and long-lasting form retention.

Another subject of the present disclosure is a method for styling keratin materials, for example human keratin materials such as the hair, which uses the compositions according to the disclosure.

Another subject of the present disclosure is the use of the compositions according to the disclosure, for example, for styling and shaping of keratin materials, such as human keratin materials such as hair.

Other features, aspects, subjects and advantages of the invention will appear even more clearly on reading the description and examples that follow.

The term “(meth)acrylic” within the meaning of the present application is understood to mean “acrylic or methacrylic”.

The branched sulphonic polyesters used in the compositions of the present disclosure are fixing polymers known in the prior art. Their structure and synthesis are described in documents WO 95/18191, WO 97/08261 and WO 97/20899.

For example, branched sulphonic polyesters may be obtained by the polycondensation of:

(a) at least one dicarboxylic acid that does not bear a sulphonic functional group,

(b) at least one diol or a mixture of a diol and a diamine,

(c) at least one monomer comprising two identical or different reactive functional groups chosen from hydroxyl, amino and carboxyl groups, and that bears, in addition, at least one sulphonic functional group, and

(d) at least one monomer comprising at least three identical or different reactive functional groups chosen from hydroxyl, amino, and carboxyl groups.

The dicarboxylic acids that form the units (a) may be aliphatic dicarboxylic acids, alicyclic dicarboxylic acids, aromatic dicarboxylic acids, and mixtures of such acids.

Mention may be made, by way of example, of 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid and isophthalic acid, and mixtures of such acids.

The diols that form the units (b) are chosen, for example, from alkanediols and polyalkylenediols, and mention may be made, by way of example, of ethylene glycol, propylene glycol, diethylene glycol, triethylene glycol, and polypropylene glycol.

The diamines capable of forming one part of the units (b) may be chosen from alkanediamines and polyoxyalkylene diamines.

The expression “sulphonic functional group” of the units (c) encompasses both the sulphonic acid functional group (—SO₃H) and the corresponding salified functional groups obtained by neutralization of the sulphonic acid functional group with a base, for example an alkali metal hydroxide.

The sulphonic functional groups may be in a form which is neutralized by an organic or inorganic base.

The units (c) are derived, for example, from dicarboxylic acids, dicarboxylic acid esters, glycols, and hydroxy acids, all bearing at least one sulphonic group, in acid and/or neutralized form, for example in neutralized form.

The units (c) bearing at least one sulphonic functional group represent, for example, from 2 to 15 mol % of the total amount of monomers.

The units (d) derived from multifunctional monomers represent, for example, from 0.1 to 40 mol % relative to the total amount of monomers.

The multifunctional monomers forming the units (d) are chosen, for example, from trimethylolethane, trimethylolpropane, glycerol, pentaerythritol, sorbitol, trimellitic anhydride, erythritol, threitol, dipentaerythritol, pyromellitic dianhydride, and dimethylpropionic acid.

The branched sulphonic polyesters may comprise, in addition to the four types of units (a) to (d) described above, units (e) derived from monomers comprising two different reactive functional groups, chosen for example from hydroxy carboxylic acids and amino carboxylic acids or mixtures thereof.

These units (e) may represent, for example, up to 40 mol % of the total amount of monomers (a), (b), (c), (d) and (e).

The branched sulphonic polymers used in the present disclosure can be obtained from a mixture of monomers in which the number of equivalents of carboxylic acid functional groups is substantially equal to the number of equivalents of hydroxyl functional groups and of amino functional groups which may be present.

The branched sulphonic polymers used in the styling compositions of the present disclosure are known and sold, for example, by Eastman. Mention may be made, as a possible commercial product, of the product sold under the name AQ 1350 by Eastman.

The composition according to the present disclosure comprises from 0.2 to 15% by weight of at least one branched sulphonic polyester relative to the total weight of the composition, such as an amount ranging from 0.5 to 10%.

The composition according to the present disclosure also comprises at least one polysaccharide thickener comprising at least one glucose unit.

For the purposes of the present disclosure, the term “thickening agent” means an agent capable, by its presence, of increasing the viscosity of the medium by at least 50 centipoise at 25° C. and at a shear rate of 1 s⁻¹. For example, the thickening agent has, at 1% in water or a 50/50 water/alcohol mixture by weight at 25° C., a viscosity of greater than 100 centipoise at a shear rate of 1 s⁻¹. These viscosities may be measured with viscometers or rheometers with cone-plate geometry.

The at least one polysaccharide thickener comprising at least one glucose unit may bechosen from celluloses, gellan gums, and derivatives thereof.

Cellulose is a β(1-4)polyacetyl of cellobiose, cellobiose being a disaccharide constituted of two glucose molecules.

The celluloses that can be used in the compositions according to the present disclosure may be chosen from non-ionic celluloses that do not comprise a hydrophobic chain and celluloses comprising at least one hydrophobic chain.

For example, the non-ionic celluloses used according to the disclosure are cellulose ethers. Examples of these celluloses include hydroxyalkyl celluloses such as hydroxyethyl celluloses or hydropropyl celluloses. They may or may not contain a fatty chain. Among the non-ionic celluloses that do not comprise a fatty chain, mention may be made of hydroxyethyl celluloses, hydropropyl celluloses, and hydroxypropyl methyl celluloses. For example a suitable hydroxypropyl methyl cellulose is METHOCEL F4M sold by Dow Chemicals (INCI name: HYDROXYPROPYLMETHYLCELLULOSE).

The celluloses modified with groups comprising at least one non-ionic fatty chains that can be used according to the present disclosure are, for example:

non-ionic hydroxyethyl celluloses modified with groups comprising at least one fatty chain such as alkyl, arylalkyl or alkylaryl groups, or mixtures thereof, and in which the alkyl groups are for example C₈-C₂₂ groups, such as the product NATROSOL PLUS GRADE 330 CS® (C₁₆ alkyls) sold by Aqualon that corresponds to the INCI name CETYLHYDROXYETHYLCELLULOSE, or the product BERMOCOLL EHM 100® sold by Berol Nobel, and

those modified with alkylphenyl polyalkylene glycol ether groups, such as the product AMERCELL POLYMER HM-1500® (nonylphenyl polyethylene glycol (15) ether) sold by Amerchol that corresponds to the INCI name NONOXYNYLHYDROXYETHYLCELLULOSE.

Gellan gum is a polysaccharide produced by aerobic fermentation of Sphingomonas elodea more commonly known as Pseudomonas elodea. This linear polysaccharide is constituted by the chaining of the following monosaccharides: D-glucose, D-glucuronic acid and L-rhamnose. In the native state, gellan gum is highly acylated.

In certain embodiments, the polysaccharide thickener is chosen from gellan gums.

In other embodiments, the gellan gum used in the compositions according to the present disclosure is chosen from an at least partially deacylated gellan gum. This at least partially deacylated gellan gum is obtained by a high-temperature alkaline treatment. A KOH or NaOH solution will, for example, be used.

The purified gellan gum sold under the trade name KELCOGEL® by Kelco is also suitable for preparing the compositions according to the invention.

The gellan gum derivatives can be products obtained by performing standard chemical reactions such as, esterifications or addition of a salt of an organic or mineral acid.

An example of a gellan gum derivative that may be used is welan gum. Welan gum is a gellan gum modified by fermentation using the Alcaligenes strain ATCC 31 555. Welan gum has a repeating pentasaccharide structure formed from a main chain constituted of D-glucose, D-glucuronic acid and L-rhamnose units onto which a pendant L-rhamnose or L-mannose unit is grafted.

The welan gum sold under the trade name KELCO CRETE® by Kelco is suitable for preparing the compositions according to the disclosure.

As other polysaccharide thickeners that can be used according to the disclosure, mention may be made of starches and derivatives thereof.

In certain embodiments, the at least one polysaccharide thickener comprising at least one glucose unit is non-ionic.

The concentration of the at least one polysaccharide thickener comprising at least one glucose unit used in the compositions according to the present disclosure can range from 0.05 to 10% by weight relative to the total weight of the composition, such as an amount ranging from 0.1 to 5% by weight, and ranging from 0.5 to 3% by weight.

In certain embodiments, the cosmetically acceptable medium is aqueous.

The cosmetic composition according to the disclosure may also comprise at least one organic solvent, such as in an amount ranging from 0.05 to 40% relative to the total weight of the composition, including for example an amount ranging from 1 to 20% by weight.

This organic solvent may be a C₂ to C₄ lower alcohol, such as ethanol, polyols and polyol ethers further such as propylene glycol, polyethylene glycol or glycerol.

The compositions according to the disclosure may also contain other cosmetically acceptable adjuvants, such as for example ionic or non-ionic surfactants, thickening agents other than the polysaccharide thickeners comprising glucose units of the invention, ethoxylated or non-ethoxylated fatty alcohols, co-thickeners, penetrants, fragrances, dyes, plasticizers, buffers, and various customary adjuvants such as waxes, volatile or non-volatile silicones that are cyclic or linear or branched, and are organomodified such as alkoxylated or modified by amine groups or are unmodified, for example silicone gums, ceramides, pseudoceramides, plant, mineral or synthetic oils, vitamins or provitamins such as panthenol, opacifiers, reducing agents, emulsifiers, preservatives, mineral fillers, pearlescent agents, flakes, sunscreens, proteins, anionic, non-ionic, cationic or amphoteric fixing polymers, moisturisers, emollients, demulcents, anti-foaming agents, antiperspirants, free-radical scavengers, bactericides, sequestrants, anti-dandruff agents, antioxidants, basifying agents, acidifying agents, and any other additive conventionally used in cosmetic compositions intended to be applied to the hair.

The surfactants that can be used in the composition according to the present disclosure may be anionic, non-ionic, amphoteric or cationic surfactants, or mixtures thereof.

Among the anionic surfactants that can be used, alone or as mixtures, in the context of the present disclosure, mention may be made for example of salts, and such as alkali metal salts further such as sodium salts, ammonium salts, amine salts, amino alcohol salts, or alkaline-earth metal salts, for example magnesium salts, of the following compounds: alkyl sulphates, alkyl ether sulphates, alkylamido ether sulphates, alkylarylpolyether sulphates, monoglyceride sulphates; alkylsulphonates, alkylamidesulphonates, alkylarylsulphonates, α-olefin sulphonates, paraffin sulphonates; alkylsulphosuccinates, alkyl ether sulphosuccinates, alkylamide sulphosuccinates; alkylsulphoacetates; acylsarcosinates; and acylglutamates, the alkyl and acyl groups of all these compounds containing from 6 to 24 carbon atoms and the aryl group such as denoting a phenyl or benzyl group.

In the context of the present disclosure, it is also possible to use C₆-C₂₄ alkyl esters of polyglycoside carboxylic acids such as alkyl glucoside citrates, polyalkyl glycoside tartrates, and polyalkyl glycoside sulphosuccinates; alkylsulphosuccinamates, acylisethionates and N-acyltaurates, the alkyl or acyl group of all these compounds containing from 12 to 20 carbon atoms. Among the anionic surfactants that may also be used, mention may also be made of acyllactylates in which the acyl group contains from 8 to 20 carbon atoms.

In addition, mention may also be made of alkyl-D-galactosideuronic acids and the salts thereof, and also polyoxyalkylenated (C₆-C₂₄)alkyl ether carboxylic acids, polyoxyalkylenated (C₆-C₂₄)alkyl(C₆-C₂₄)aryl ether carboxylic acids, polyoxyalkylenated (C₆-C₂₄)alkylamido ether carboxylic acids and salts thereof, for example those containing from 2 to 50 ethylene oxide groups, and mixtures thereof.

The non-ionic surfactants that may be used in the context of the present invention are, themselves also, compounds that are well known per se (see for example in this respect “Handbook of Surfactants” by M. R. Porter, published by Blackie & Son (Glasgow and London), 1991, pp. 116-178). They can be chosen for example from polyethoxylated, polypropoxylated or polyglycerolated fatty acids, (C₁-C₂₀)alkylphenols, α-diols or alcohols having a fatty chain containing, for example, 8 to 18 carbon atoms, it being possible for the number of ethylene oxide or propylene oxide groups to range for example from 2 to 50 and for the number of glycerol groups to range such as from 2 to 30. Mention may also be made of copolymers of ethylene oxide and of propylene oxide, condensates of ethylene oxide and of propylene oxide with fatty alcohols; polyethoxylated fatty amides for example having from 2 to 30 mol of ethylene oxide, polyglycerolated fatty amides containing on average 1 to 5, and for example 1.5 to 4, glycerol groups; polyethoxylated fatty amines such as having 2 to 30 mol of ethylene oxide; ethoxylated fatty acid esters of sorbitan having from 2 to 30 mol of ethylene oxide; fatty acid esters of sucrose, fatty acid esters of polyethylene glycol, (C₆-C₂₄)alkylpolyglucosides, N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

The amphoteric surfactants that are suitable for use in the present disclosure may for example be aliphatic secondary or tertiary amine derivatives, in which the aliphatic group is a linear or branched chain containing 8 to 22 carbon atoms and containing at least one water-solubilizing anionic group, such as, for example, a carboxylate, sulphonate, sulphate, phosphate or phosphonate group; mention may also be made of (C₈-C₂₀)alkylbetaines, sulphobetaines, (C₈-C₂₀)alkylamido(C₆-C₈)alkylbetaines or (C₈-C₂₀)alkylamido(C₆-C₈)alkylsulphobetaines; and mixtures thereof.

Among the amine derivatives that may be mentioned are the products sold under the name MIRANOL®, as described for example in U.S. Pat. Nos. 2,528,378 and 2,781,354 and classified in the CTFA dictionary, 3rd edition, 1982, under the names amphocarboxyglycinate and amphocarboxypropionate, and having the respective structures (1) and (2):

R₂—CONHCH₂CH₂—N⁺(R₃)(R₄)(CH₂COO⁻) (1)

in which:

R₂ represents an alkyl group derived from an acid R₂—COOH present in hydrolysed coconut oil, or a heptyl, nonyl or undecyl group,

R₃ represents a β-hydroxyethyl group, and

R₄ represents a carboxymethyl group; and

R₂—CONHCH₂CH₂—N(B)(C) (2)

in which:

B represents —CH₂CH₂OX′,

C represents —(CH₂)_{z}—Y′, with z=1 or 2,

X′ represents the —CH₂CH₂—COOH group or a hydrogen atom,

Y′ represents —COON or the —CH₂—CHOH—SO₃H group,

R₂ represents the alkyl group of an acid R₂′-COOH present in coconut oil or in hydrolysed linseed oil, an alkyl group, for example a C₁₇ group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name MIRANOL® C2M concentrate by the company Rhodia.

Among the amphoteric surfactants that may be used are (C₈-C₂₀)alkylbetaines such as cocobetaine, (C₈-C₂₀)alkylamido(C₆-C₈)alkylbetaines such as cocamidobetaine, and alkylamphodiacetates, for instance disodium cocoamphodiacetate, and mixtures thereof.

The composition according to the present disclosure may also comprise at least one cationic surfactant that are well known per se, such as primary, secondary or tertiary fatty amine salts, optionally polyoxyalkylenated; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

The non-ionic, amphoteric and cationic surfactants described above may be used alone or as mixtures and the amount thereof can range from 0.01% to 30% by weight relative to the total weight of the composition, such as an amount ranging from 0.05% to 20% by weight and from 0.1% to 10% by weight.

The additional gelling agents and/or thickeners suitable for the compositions of the present disclosure, other than the at least one polysaccharide thickener comprising at least one glucose unit, are well known in the art and may be chosen from poly(oxyalkylene) glycols, poly(oxyalkylene) glycol esters, alginates, biosaccharides, starch derivatives, natural gums such as xanthan gun, carob bean gum, scleroglucans, derivatives of chitin and of chitosan, carrageenans, clays, and mixtures thereof.

By way of example of gelling agents, including those that are in the aqueous phase, mention may be made of SEPIGEL® 305 sold by the company SEPPIC, FUCOGEL® 1000 PP sold by the company Solabia, SYNTHALEN® K sold by the company 3VSA, LUVISKOL® VA 64 P sold by BASF, HOSTACERIN® AMPS sold by Clariant, LUBRAGEL® MS sold by Guardian, SATIAGEL® KSO sold by Degussa and KELTROL® sold by the company Kelco, SALCARE SC 95 or SC 96 sold by Ciba.

The additional gelling agents are generally present in an amount ranging from 0.05 to 15% by weight of the composition, such as an amount ranging from 0.5 to 10%.

The silicones that may be used as additives in the cosmetic compositions of the present disclosure are volatile or non-volatile, cyclic, linear or branched silicones, optionally modified with organic groups. In certain embodiments, these silicones can have a viscosity ranging from 5×10⁻⁶ to 2.5 m²/s at 25° C., such as from 1×10⁻⁵ to 1 m²/s.

The silicones that can be used in accordance with the present disclosure may be soluble or insoluble in the composition and, for example, may be polyorgano-siloxanes that are insoluble in the composition of the present disclosure. They may be in the form of oils, waxes, resins, or gums.

The organopolysiloxanes are defined in greater detail in Walter Noll's “Chemistry and Technology of Silicones” (1968), Academic Press. They can be volatile or non-volatile.

When they are volatile, the silicones are, for example, chosen from those having a boiling point ranging from 60° C. to 260° C. Suitable silicones include cyclic silicones containing from 3 to 7 silicon atoms, such as from 4 to 5 silicon atoms. These include, for example, octamethylcyclotetrasiloxane sold under the name VOLATILE SILICONE® 7207 by Union Carbide or SILIBIONE® 70045 V2 by Rhodia, decamethylcyclopentasiloxane sold under the name VOLATILE SILICONE® 7158 by Union Carbide, and SILIBIONE® 70045 V5 by Rhodia, and mixtures thereof.

Mention may also be made of cyclocopolymers of the dimethylsiloxane/methylalkylsiloxane type, such as SILICONE VOLATILE® FZ 3109 sold by the company Union Carbide, of formula:

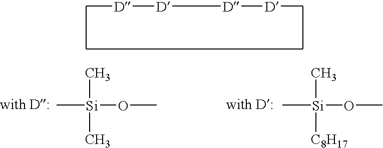

Mention may also be made of mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane

Suitable silicones also include linear volatile silicones containing 2 to 9 silicon atoms and having a viscosity of less than or equal to 5×10⁻⁶ m²/s at 25° C. An example is decamethyltetrasiloxane sold for example under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, such as polyalkylsiloxanes, polyarylsiloxanes, polyalkylarylsiloxanes, silicone gums and resins, polyorganosiloxanes modified with organofunctional groups, and mixtures thereof, may be used.

These silicones may for example be chosen from polyalkylsiloxanes, among which mention may be made of polydimethylsiloxanes containing trimethylsilyl end groups. The viscosity of the silicones is measured at 25° C. according to ASTM 445 standard Appendix C.

Among these polyalkylsiloxanes, mention may be made, in a non-limiting manner, of the following commercial products:

the SILIBIONE® oils of the 47 and 70 047 series or the MIRASIL® oils sold by Rhodia, such as, for example, the oil 70 047 V 500 000;

the oils of the MIRASIL® series sold by the company Rhodia;

the oils of the 200 series from the company Dow Corning, such as, DC200 with a viscosity of 60 000 mm²/s; and

the VISCASIL® oils from General Electric and certain oils of the SF series (SF 96, SF 18) from General Electric.

Mention may also be made of polymethylsiloxanes containing dimethylsilanol end groups, known by the name dimethiconol (CTFA), such as the oils of the 48 series from the company Rhodia.

In this category of polyalkylsiloxanes, mention may also be made of the products sold under the names ABIL WAX® 9800 and 9801 by the company Goldschmidt, which are poly(C₁-C₂₀)alkylsiloxanes.

The polyalkylarylsiloxanes are chosen for example from linear and/or branched polydimethyl/methylphenylsiloxanes and polydimethyl/diphenylsiloxanes with a viscosity of ranging from 1×10⁻⁵ to 5×10⁻² m²/s at 25° C.

Among these polyalkylarylsiloxanes, mention may be made, by way of example, of the products sold under the following names:

the SILIBIONE® oils of the 70 641 series from Rhodia;

the oils of the RHODORSIL® 70 633 and 763 series from Rhodia;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000; and

certain oils of the SF series from General Electric, such as SF 1023, SF 1154, SF 1250 and SF 1265.

The silicone gums that can be used in accordance with the disclosure are, for example, polyorganosiloxanes having high number-average molecular weights ranging from 200,000 to 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made for example of the following products:

polydimethylsiloxane gums,

polydimethylsiloxane/methylvinylsiloxane gums,

polydimethylsiloxane/diphenylsiloxane gums,

polydimethylsiloxane/phenylmethylsiloxane gums, and

polydimethylsiloxane/diphenylsiloxane/methylvinylsiloxane gums.

Products that can be used for example in accordance with the disclosure are mixtures such as:

mixtures formed from a polydimethylsiloxane hydroxylated at the end of the chain, or dimethiconol (CTFA) and from a cyclic polydimethylsiloxane also called cyclomethicone (CTFA), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane; and

mixtures of two PDMSs of different viscosities, and such as a PDMS gum and a PDMS oil, further such as the product SF 1236 from the company General Electric. The product SF 1236 is the mixture of an SE 30 gum defined above, having a viscosity of 20 m²/s, and an SF 96 oil, with a viscosity of 5×10⁻⁶ m²/s. This product for example contains 15% SE 30 gum and 85% SF 96 oil.

The organopolysiloxane resins that can be used in accordance with the disclosure can be crosslinked siloxane systems containing the following units:

R₂SiO_{2/2}, R₃SiO_{1/2}, RSiO_{3/2} and SiO_{4/2}

in which R represents a hydrocarbon-based group containing 1 to 16 carbon atoms or a phenyl group. Among these products, for example are the ones in which R denotes a C₁-C₄ lower alkyl group, such as methyl, or a phenyl group.

Among these resins, mention may be made of the product sold under the name Dow Corning 593 or those sold under the names Silicone Fluid SS 4230 and SS 4267 by the company General Electric, which are silicones of dimethyl/trimethylsiloxane structure.

Mention may also be made of the trimethylsiloxysilicate type resins sold for example under the names X22-4914, X21-5034 and X21-5037 by the company Shin-Etsu.

The organomodified silicones that can be used in accordance with the disclosure are silicones as defined above and containing in their structure at least one organofunctional group attached via a hydrocarbon-based group.

Among the organomodified silicones, mention may be made of polyorganosiloxanes comprising:

polyethyleneoxy and/or polypropyleneoxy groups optionally containing C₆-C₂₄ alkyl groups, such as the products known as dimethicone copolyol sold by the company Dow Corning under the name DC 1248 or the oils SILWET® L 722, L 7500, L 77, L 711 from the company Union Carbide and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 or Dow Corning 2-8299 by the company Dow Corning or the product sold under the name BELSIL ADM LOG 1 by the company Wacker. The substituted amine groups are, for example, C₁-C₄ aminoalkyl groups;

thiol groups such as the products sold under the names GP 72A and GP 71 from Genesee;

alkoxylated groups such as the product sold under the name Silicone Copolymer F-755 by SWS Silicones and ABIL WAX® 2428, 2434 and 2440 by the company Goldschmidt;

hydroxylated groups such as the polyorganosiloxanes containing a hydroxyalkyl functional group, described in French patent application FR-A-8 516 334;

alkoxyalkyl groups such as, for example, the polyorganosiloxanes described in U.S. Pat. No. 4,957,732;

anionic groups of carboxylic type, such as, for example, in the products described in patent EP 186 507 from the company Chisso Corporation, or of alkylcarboxylic type, such as those present in the product X-22-3701 E from the company Shin-Etsu; 2-hydroxyalkyl sulphonate; 2-hydroxyalkyl thiosulphate such as the products sold by the company Goldschmidt under the names ABIL® S201 and ABIL® S255; and

hydroxyacrylamino groups, such as the polyorganosiloxanes described in patent application EP 342 834. Mention may be made, for example, of the product Q2-8413 from the company Dow Corning.

The silicones as described above may be used, alone or as a mixture, in an amount ranging from 0.01% to 20% by weight relative to the total weight of the composition, such as an amount ranging from 0.1% to 5% by weight.

The compositions of the disclosure may also comprise non-silicone fatty substances such as mineral, plant, animal and synthetic oils, waxes, fatty esters, ethoxylated or non-ethoxylated fatty alcohols, and fatty acids.

As oils that can be used in the composition of the disclosure, examples that may be mentioned include:

hydrocarbon-based oils of animal origin, such as perhydrosqualene;

hydrocarbon-based oils of plant origin, such as liquid fatty acid triglycerides containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stéarineries Dubois or those sold under the names MIGLYOL® 810, 812 and 818 by the company Dynamit Nobel, jojoba oil, and shea butter oil;

linear or branched hydrocarbons of mineral or synthetic origin, such as volatile or non-volatile liquid paraffins, and derivatives thereof, petroleum jelly, polydecenes, hydrogenated polyisobutene such as PARLEAM®; isoparaffins, for instance isohexadecane and isodecane;

partially hydrocarbon-based and/or silicone-based fluoro oils, for instance those described in document JP-A-2-295 912; fluoro oils that may also be mentioned include perfluoromethylcyclopentane and perfluoro-1,3-dimethylcyclohexane, sold under the names FLUTEC® PC1 and FLUTEC® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name FORALKYL® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The at least one wax may be chosen, for example, from carnauba wax, candelilla wax, esparto grass wax, paraffin wax, ozokerite, plant waxes such as olive wax, rice wax, hydrogenated jojoba wax, or the absolute waxes of flowers such as the essential wax of blackcurrant blossom sold by the company Berlin (France), animal waxes, for instance beeswaxes or modified beeswaxes (cerabellina); other waxes or waxy starting materials that can be used according to the disclosure are for example marine waxes such as the product sold by the company Sophim under the reference M82, and polyethylene waxes or polyolefin waxes in general.

The saturated or unsaturated fatty acids are, for example, chosen from myristic acid, palmitic acid, stearic acid, behenic acid, oleic acid, linoleic acid, linolenic acid, and isostearic acid.

The fatty esters are, for example, carboxylic acid esters, such as mono-, di-, tri-, or tetracarboxylic esters.

The carboxylic acid esters are, for example, esters of saturated or unsaturated, linear or branched C₁-C₂₆ aliphatic acids and of saturated or unsaturated, linear or branched C₁-C₂₆ aliphatic alcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate.

Esters of C₄-C₂₂ dicarboxylic or tricarboxylic acids and of C₁-C₂₂ alcohols and esters of mono-, di-, or tricarboxylic acids and of C₂-C₂₆ di-, tri-, tetra-, or pentahydroxy alcohols may also be used.

The following esters may for example be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate, and neopentyl glycol diheptanoate. The esters mentioned above being different from the esters of formula (I).

Among the esters mentioned above, it is possible to use ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate, or cetyl octanoate.

As fatty alcohols, mention may be made of linear or branched, saturated or unsaturated fatty alcohols containing from 8 to 26 carbon atoms, for instance cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol.

The fatty substances in general are present in an amount ranging from 0.1 to 50% by weight relative to the total weight of the composition; such as an amount ranging from 1 to 30%, and an amount ranging from 2 to 20% by weight.

As indicated previously, the compositions may comprise at least one additional fixing polymer other than the polymers of the invention. The expression “fixing polymer” is understood within the meaning of the present invention to mean any polymer that makes it possible to give a shape to the hair or to hold the hair in a given shape.

All the anionic, cationic, amphoteric and non-ionic fixing polymers other than branched sulphonic polymers and mixtures thereof used in the art may be used as additional fixing polymers in the compositions according to the present application.

The fixing polymers may be soluble in the cosmetically acceptable medium or insoluble in this same medium and used in this case in the form of dispersions of solid or liquid particles of polymer (latex or pseudolatex).

The anionic fixing polymers generally used are polymers comprising groups derived from carboxylic, sulphonic or phosphoric acid and have a number-average molecular weight ranging from 500 to 5,000,000.

The carboxylic groups are provided by unsaturated carboxylic monoacid or diacid monomers such as those corresponding to the formula:

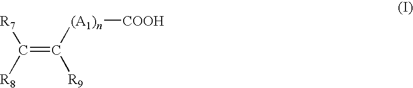

in which n is an integer ranging from 0 to 10, A₁ denotes a methylene group, optionally connected to the carbon atom of the unsaturated group or to the neighboring methylene group when n is greater than 1, via a heteroatom such as oxygen or sulphur, R₇ denotes a hydrogen atom, or a phenyl or benzyl group, R₈ denotes a hydrogen atom or a lower alkyl or carboxyl group, R₉ denotes a hydrogen atom, a lower alkyl group or a —CH₂—COON, phenyl, or benzyl group.

In the aforementioned formula, a lower alkyl group for example denotes a group having 1 to 4 carbon atoms such as methyl and ethyl groups.

Suitable anionic fixing polymers containing carboxylic groups according to the disclosure include:

A) The homopolymers or copolymers of acrylic or methacrylic acid or salts thereof such as the products sold under the names VERSICOL® E or K by the company Allied Colloid and ULTRAHOLD® by BASF, copolymers of acrylic acid and of acrylamide sold in the form of their sodium salts under the names RETEN 421, 423 or 425 by the company Hercules, the sodium salts of polyhydroxycarboxylic acids.

B) Copolymers of acrylic or methacrylic acid with a monoethylenic monomer such as ethylene, styrene, vinyl esters, acrylic, or methacrylic acid esters, optionally grafted onto a polyalkylene glycol such as polyethylene glycol and optionally crosslinked. Such polymers are described for example in French patent 1 222 944 and German patent application 2 330 956, the copolymers of this type comprising an optionally N-alkylated and/or hydroxyalkylated acrylamide unit in their chain as described for example in Luxembourg patent applications 75370 and 75371 or sold under the name Quadramer by the company American Cyanamid. Mention may also be made of the acrylic acid/ethyl acrylate/N-tert-butylacrylamide terpolymers such as ULTRAHOLD STRONG sold by the company BASF. Mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name ACRYLIDONE® LM and methacrylic acid/ethyl acrylate/tert-butyl acrylate terpolymers such as the product sold under the name LUVIMER® 100 P by the company BASF. Mention may also be made of methacrylic acid/acrylic acid/ethyl acrylate/methyl methacrylate copolymers as an aqueous dispersion, sold under the name AMERHOLD® DR 25 by the company Amerchol.

C) Crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon chain such as those containing at least 5 carbon atoms, it being possible for these polymers optionally to be grafted or crosslinked, or alternatively another vinyl, allyl or methallyl ester monomer of an α- or β-cyclic carboxylic acid. Such polymers are described, inter alia, in French patents 1 222 944, 1 580 545, 2 265 782, 2 265 781, 1 564 110 and 2 439 798. Commercial products falling into this class are the resins 28-29-30, 26-13-14 and 28-13-10 sold by the company National Starch.

D) Copolymers of C₄-C₈ monounsaturated carboxylic acids or anhydrides chosen from:

copolymers comprising (i) at least one maleic, fumaric, or itaconic acid or anhydride and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described for example in U.S. Pat. Nos. 2,047,398, 2,723,248 and 2,102,113 and GB patent 839,805. Commercial products are for example those sold under the names GANTREZ® AN or ES by the company ISP,

copolymers comprising (i) at least one maleic, citraconic, or itaconic anhydride unit and (ii) at least one monomerschosen from allyl or methallyl esters optionally comprising at least one acrylamide, methacrylamide, α-olefin, acrylic, or methacrylic ester, acrylic or methacrylic acid or vinylpyrrolidone groups in their chain, the anhydride functions of these copolymers optionally being monoesterified or monoamidated. These polymers are described, for example, in French patents 2 350 384 and 2 357 241 by the Applicant.

E) Polyacrylamides comprising carboxylate groups.

F) Homopolymers and copolymers comprising at least one sulphonic group such as polymers comprising vinylsulphonic, styrenesulphonic, naphthalenesulphonic or acrylamidoalkylsulphonic units, different from the branched sulphonic polyesters of the disclosure.

The homopolymers and copolymers comprising at least one sulphonic group polymers can be chosen for example from:

polyvinylsulphonic acid salts having a molecular weight of approximately ranging from 1,000 to 100,000, and also the copolymers with an unsaturated comonomer such as acrylic or methacrylic acids and their esters, and also acrylamide or its derivatives, vinyl ethers, and vinylpyrrolidone;

polystyrenesulphonic acid salts such as the sodium salts that are sold for example under the names FLEXAN® 500 and FLEXAN® 130 by National Starch. These compounds are described in patent FR 2 198 719; and

polyacrylamidesuiphonic acid salts, such as those mentioned in U.S. Pat. No. 4,128,631 for example polyacrylamidoethylpropanesulphonic acid sold under the name COSMEDIA POLYMER HSP 1180 by Henkel.

As another anionic fixing polymer that can be used according to the disclosure, mention may be made of the branched block anionic polymer sold under the name FIXATE G-100 by the company Noveon.

According to the disclosure, the anionic fixing polymers may be chosen from copolymers of acrylic acid or of acrylic esters, such as the acrylic acid/ethyl acrylate/N-tert-butylacrylamide terpolymers sold for example under the name ULTRAHOLD® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold for example under the name RESIN 28-29-30 by the company National Starch, polymers derived from maleic, fumaric, or itaconic acids or anhydrides with vinyl esters, vinyl ethers, vinyl halides, phenylvinyl derivatives, and acrylic acid and esters thereof, such as the methyl vinyl ether/monoesterified maleic anhydride copolymers sold, for example, under the name GANTREZ® by the company ISP, the copolymers of methacrylic acid and of methyl methacrylate sold under the name EUDRAGIT® by the company Rohm Pharma, the copolymers of methacrylic acid and of ethyl acrylate sold under the name LUVIMER® MAEX or MAE by the company BASF, the vinyl acetate/crotonic acid copolymers sold under the name LUVISET CA 66 by the company BASF, the vinyl acetate/crotonic acid copolymers grafted with polyethylene glycol sold under the name ARISTOFLEX® A by the company BASF, and the polymer sold under the name FIXATE G-100 by the company Noveon.

Among the anionic fixing polymers mentioned above, certain embodiments of the present disclosure use the monoesterified methyl vinyl ether/maleic anhydride copolymers sold under the name GANTREZ® ES 425 by the company ISP, the acrylic acid/ethyl acrylate/N-tert-butylacrylamide terpolymers sold under the name ULTRAHOLD® Strong by the company BASF, the copolymers of methacrylic acid and of methylmethacrylate sold under the name EUDRAGIT® L by the company Rohm Pharma, the vinylacetate/vinyl tert-butylbenzoate/crotonic acid terpolymers and the crotonic acid/vinylacetate/vinyl neododecanoate terpolymers sold under the name RESIN 28-29-30 by the company National Starch, the copolymers of methacrylic acid and of ethylacrylate sold under the name LUVIMER® MAEX or MAE by BASF, the vinylpyrrolidone/acrylic acid/laurylmethacrylate terpolymers sold under the name ACRYLIDONE® LM by the company ISP, and the polymer sold under the name FIXATE G-100 by the company Noveon.

The cationic fixing film-forming polymers that can be used according to the present disclosure may be chosen from polymers comprising primary, secondary, tertiary, and/or quaternary amine groups forming part of the polymer chain or directly attached thereto, and having a molecular weight ranging from 500 to about 5,000,000 and such as ranging from 1,000 to 3,000,000.

Among these polymers, mention may be for example made of the following cationic polymers:

(1) Homopolymers or copolymers derived from acrylic or methacrylic esters or amides and comprising at least one of the units of the following formulae:

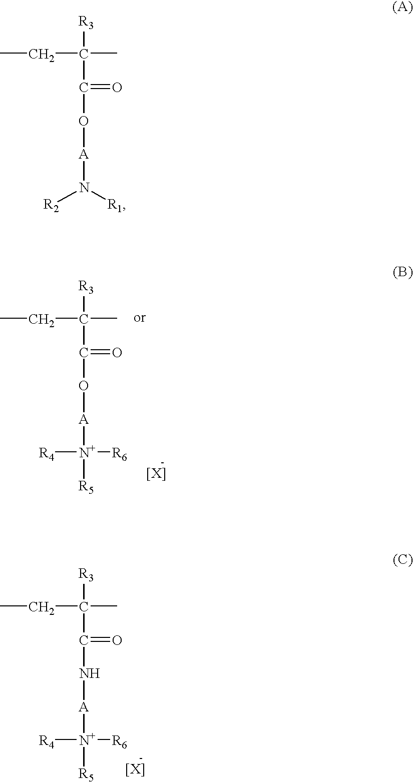

in which:

R₃ denotes a hydrogen atom or a CH₃ radical;

A is a linear or branched alkyl group comprising 1 to 6 carbon atoms or a hydroxyalkyl group comprising 1 to 4 carbon atoms;

R₄, R₅ and R₆, which may be identical or different, represent an alkyl group having from 1 to 18 carbon atoms or a benzyl radical;

R₁ and R₂, which may be identical or different, each represent a hydrogen atom or an alkyl group having from 1 to 6 carbon atoms; and

X denotes a methosulphate anion or a halide such as chloride or bromide.

The copolymers of the family (1) also contain at least one unit derived from comonomers that may be chosen from the family of acrylamides, methacrylamides, diacetone acrylamides, acrylamides and methacrylamides substituted on the nitrogen with lower (C₁₋₄) alkyl groups, groups derived from acrylic or methacrylic acids or esters thereof, vinyllactams such as vinylpyrrolidone or vinylcaprolactam, and vinyl esters.

Thus, among these copolymers of the family (1), mention may be made of:

copolymers of acrylamide and of dimethylaminoethyl methacrylate quaternized with dimethyl sulphate or with a dimethyl halide, such as the product sold under the name HEROFLOC® by the company Hercules,

copolymers of acrylamide and of methacryloyloxyethyltrimethylammonium chloride, described, for example, in patent application EP-A-080 976 and sold under the name BINA QUAT P 100 by the company Ciba Geigy,

copolymers of acrylamide and of methacryloyloxyethyltrimethylammonium methosulphate, such as the product sold under the name RETEN by the company Hercules,

quaternized or non-quaternized vinylpyrrolidone/dialkylaminoalkyl acrylate or methacrylate copolymers, such as the products sold under the name “GAFQUAT®” by the company ISP, such as, for example, “GAFQUAT® 734” or “GAFQUAT® 755”, or alternatively the products known as “COPOLYMER® 845, 958 and 937”. These polymers are described in detail in French patents 2 077 143 and 2 393 573,

fatty-chain polymers containing a vinylpyrrolidone unit, such as the products sold under the name STYLEZE W20 and STYLEZE W10 by the company ISP,

dimethylaminoethyl methacrylate/vinylcaprolactam/vinylpyrrolidone terpolymers, such as the product sold under the name GAFFIX VC 713 by the company ISP, and

quaternized vinylpyrrolidone/dimethylaminopropylmethacrylamide copolymers, such as the products sold under the name “GAFQUAT® HS100” by the company ISP.

(2) Non-cellulosic cationic polysaccharides, for example containing quaternary ammonium, such as those described in U.S. Pat. Nos. 3,589,578 and 4,031,307, such as guar gums containing trialkylammonium cationic groups. Such products are sold for example under the trade names JAGUAR C13 S, JAGUAR C 15 and JAGUAR C 17 by the company Meyhall.

(3) Quaternary copolymers of vinylpyrrolidone and of vinylimidazole.

(4) Chitosans or salts thereof; the salts that can be used are, for example, chitosan acetate, lactate, glutamate, gluconate, or pyrrolidonecarboxylate.
Among these compounds, mention may be made of chitosan having a degree of deacetylation of 90.5% by weight, sold under the name KYTAN BRUT STANDARD by the company Aber Technologies, and chitosan pyrrolidonecarboxylate sold under the name KYATMER® PC by the company Amerchol.

(5) Cationic cellulose derivatives such as copolymers of cellulose or of cellulose derivatives grafted with a water-soluble monomer comprising a quaternary ammonium, and disclosed for example in U.S. Pat. No. 4,131,576, such as hydroxyalkylcelluloses, for inst^{a}nce hydroxymethyl-, hydroxyethyl- or hydroxypropylcelluloses grafted for example with a methacryloyloxyethyltrimethylammonium, methacrylamidopropyltrimethylammonium, or dimethyldiallylammonium salt.

The products sold corresponding to this definition are, for example, the products sold under the name “CELQUAT L 200” and “CELQUAT H 100” by the company National Starch.

The amphoteric fixing polymers that can be used in accordance with the disclosure can be chosen from polymers comprising units B and C distributed randomly in the polymer chain, in which B denotes a unit derived from a monomer comprising at least one basic nitrogen atom and C denotes a unit derived from an acid monomer comprising one or more carboxylic or sulphonic groups, or alternatively B and C can denote groups derived from carboxybetaine or sulphobetaine zwitterionic monomers.

B and C can also denote a cationic polymer chain comprising primary, secondary, tertiary, or quaternary amine groups, in which at least one of the amine groups bears a carboxylic or sulphonic group connected via a hydrocarbon group or alternatively B and C form part of a chain of a polymer containing an α,β-dicarboxylic ethylene unit in which one of the carboxylic groups has been made to react with a polyamine comprising at least one primary or secondary amine group.

The amphoteric fixing polymers corresponding to the definition given above can be chosen from the following polymers:
(1) Copolymers having acidic vinyl units and basic vinyl units, such as those resulting from the copolymerization of a monomer derived from a vinyl compound bearing a carboxylic group such as, acrylic acid, methacrylic acid, maleic acid, α-chloroacrylic acid, and a basic monomer derived from a substituted vinyl compound containing at least one basic atom, such as, dialkylaminoalkyl methacrylates and acrylates, dialkylaminoalkylmethacrylamides, and -acrylamides. Such compounds are described in U.S. Pat. No. 3,836,537.

(2) Polymers comprising units derived from:

a) at least one monomer chosen from acrylamides and methacrylamides substituted on the nitrogen atom with an alkyl group,

b) at least one acidic comonomer containing at least one reactive carboxylic group, and

c) at least one basic comonomer such as esters containing primary, secondary, tertiary, and quaternary amine substituents of acrylic and methacrylic acids and the product of quaternization of dimethylaminoethyl methacrylate with dimethyl or diethyl sulphate.

The N-substituted acrylamides or methacrylamides that may be for example according to the disclosure are compounds in which the alkyl groups contain from 2 to 12 carbon atoms such as N-ethylacrylamide, N-tert-butylacrylamide, N-tert-octylacryl-amide, N-octylacrylamide, N-decylacrylamide, N-dodecylacrylamide, and the corresponding methacrylamides.

The acidic comonomers are chosen for example from acrylic acid, methacrylic acid, crotonic acid, itaconic acid, maleic acid, and fumaric acid and alkyl monoesters, having 1 to 4 carbon atoms, of maleic or fumaric acids or anhydrides.

For example, the basic comonomers are aminoethyl, butylaminoethyl, N,N′-dimethylaminoethyl, and N-tert-butylaminoethyl methacrylates.
The copolymers whose CTFA (4th edition, 1991) name is octylacryl-amide/acrylates/butylaminoethyl methacrylate copolymer, such as the products sold under the name AMPHOMER® or LOVOCRYL® 47 by the company National Starch, are for example used.

(3) Crosslinked and acylated polyamino amides partially or totally derived from polyamino amides of general formula:

CO—R₁₀—CO—Z (II)

in which R₁₀ represents a divalent group derived from a saturated dicarboxylic acid, a mono- or dicarboxylic aliphatic acid containing an ethylenic double bond, an ester of a lower alkanol, having 1 to 6 carbon atoms, of these acids, or a group derived from the addition of any one of said acids to a bis(primary) or bis(secondary) amine, and Z denotes a group derived from a bis(primary), mono- or bis(secondary) polyalkylene-polyamine and may represents:

a) in proportions of from 60 to 100 mol %, the group:

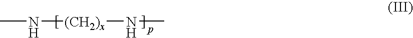

where x=2 and p=2 or 3, or alternatively x=3 and p=2

this group being derived from diethylenetriamine, from triethylenetetraamine or from dipropylenetriamine;

b) in proportions of from 0 to 40 mol %, the group (X) above in which x=2 and p=1 and which is derived from ethylenediamine, or the group derived from piperazine:

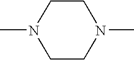

c) in proportions of from 0 to 20 mol %, the —NH—(CH₂)₆—NH— group being derived from hexamethylenediamine,

these polyamino amides being crosslinked by addition reaction of a difunctional crosslinking agent chosen from epihalohydrins, diepoxides, dianhydrides and bis-unsaturated derivatives, using ranging from 0.025 to 0.35 mol of crosslinking agent per amine group of the polyamino amide and acylated by the action of acrylic acid, chloroacetic acid or an alkane sultone, or salts thereof.

The saturated carboxylic acids are for example chosen from acids having 6 to 10 carbon atoms, such as adipic acid, 2,2,4-trimethyladipic acid, and 2,4,4-trimethyladipic acid, terephthalic acid, acids containing an ethylenic double bond such as, for example, acrylic acid, methacrylic acid, and itaconic acid.
The alkane sultones used in the acylation are for example propane sultone or butane sultone; the salts of the acylating agents may be the sodium or potassium salts.

(4) Polymers comprising zwitterionic units of formula:

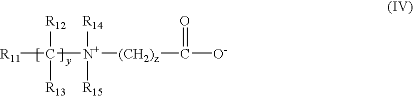

in which R₁₁ denotes a polymerizable unsaturated group such as an acrylate, methacrylate, acrylamide, or methacrylamide group, y and z represent an integer from 1 to 3, R₁₂ and R₁₃ represent a hydrogen atom, a methyl, ethyl, or propyl group, R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.
By way of example, mention may be made of the copolymers of methyl methacrylate/methyl dimethylcarboxymethylammonioethyl methacrylate such as the product sold under the name DIAFORMER Z301 by the company Sandoz.

(5) Polymers derived from chitosan comprising monomer units corresponding to the following formulae:

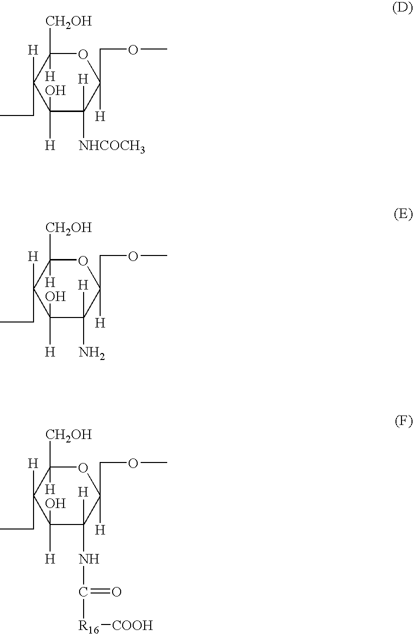

the unit (D) being present in proportions ranging from 0 to 30%, the unit (E) in proportions ranging form 5% to 50% and the unit (F) in proportions ranging from 30% to 90%, it being understood that, in this unit (F), R₁₆ represents a group of formula:

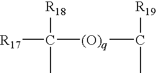

in which, if q=0, R₁₇, R₁₈ and R₁₉, which may be identical or different, each represent a hydrogen atom, a methyl, hydroxyl, acetoxy, or amino residue, a monoalkylamine residue or a dialkylamine residue that are optionally interrupted by at least one nitrogen atom and/or optionally substituted with at least one functional group chosen fromamine, hydroxyl, carboxyl, alkylthio, or sulphonic groups, an alkylthio residue in which the alkyl group bears an amino residue, at least one of the groups R₁₇, R₁₈ and R₁₉ being, in this case, a hydrogen atom;

or, if q=1, R₁₇, R₁₈ and R₁₉ each represent a hydrogen atom, as well as the salts formed by these compounds with bases or acids.

(6) Polymers with units corresponding to the general formula (XII) are described, for example, in French patent 1 400 366:

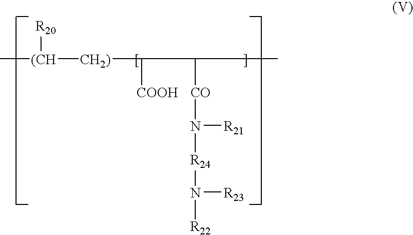

in which R₂₀ represents a hydrogen atom, a CH₃O, CH₃CH₂O, or phenyl group, R₂₁ denotes a hydrogen atom or a lower alkyl group such as methyl or ethyl, R₂₂ denotes a hydrogen atom or a C₁₋₆ lower alkyl group such as methyl or ethyl, R₂₃ denotes a C₁₋₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above.

(7) Polymers derived from the N-carboxyalkylation of chitosan, such as N-carboxymethylchitosan or N-carboxybutylchitosan sold under the name “EVALSAN” by the company Jan Dekker.

(8) Amphoteric polymers of the type -D-X-D-X chosen from:

a) polymers obtained by the action of chloroacetic acid or sodium chloroacetate on compounds comprising at least one unit of formula:

-D-X-D-X-D- (VI)

where D denotes a group

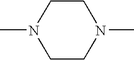

and X denotes the symbol E or E′, E or E′, which may be identical or different, denote a divalent group that is an alkylene group with a straight or branched chain containing up to 7 carbon atoms in the main chain, which is unsubstituted or substituted with hydroxyl groups and which can comprise, in addition to oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester, and/or urethane groups;

b) polymers of formula:

-D-X-D-X- (VII)

where D denotes a group

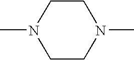

and X denotes the symbol E or E′ and at least once E′; E having the meaning given above and E′ is a divalent group that is an alkylene group with a straight or branched chain having up to 7 carbon atoms in the main chain, which is unsubstituted or substituted with at least one hydroxyl group and containing at least one nitrogen atom, the nitrogen atom being substituted with an alkyl chain that is optionally interrupted by an oxygen atom and necessarily comprising at least one carboxyl function or at least one hydroxyl function and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

In certain embodiments, the amphoteric fixing polymers mentioned above can be chosen from those of family (3), such as the copolymers whose CTFA name is octylacrylamide/acrylates/butylaminoethyl methacrylate copolymer, such as the products sold under the names AMPHOMER®, AMPHOMER® LV 71 or LOVOCRYL® 47 by the company National Starch, and those of family (4) such as the copolymers of methyl methacrylate/methyl dimethylcarboxymethylammonioethyl methacrylate, sold, for example, under the name DIAFORMER Z301 by the company Sandoz.

The non-ionic fixing polymers that may be used according to the present disclosure are chosen, for example, from:

polyalkyloxazolines;

vinyl acetate homopolymers;

vinyl acetate copolymers, for instance copolymers of vinyl acetate and of acrylic ester, copolymers of vinyl acetate and of ethylene, or copolymers of vinyl acetate and of maleic ester, for example of dibutyl maleate;

homopolymers and copolymers of acrylic esters, for instance copolymers of alkyl acrylates and of alkyl methacrylates, such as the products sold by the company Rohm & Haas under the names PRIMAL® AC-261 K and EUDRAGIT® NE 30 D, by the company BASF under the name 8845, or by the company Hoechst under the name APPRETAN® N9212;

copolymers of acrylonitrile and of a non-ionic monomer chosen, for example, from butadiene and alkyl (meth)acrylates; mention may be made of the products sold under the name CJ 0601 B by the company Rohm & Haas;

styrene homopolymers;

styrene copolymers, for instance copolymers of styrene and of an alkyl (meth)acrylate, such as the products MOWILITH® LDM 6911, MOWILITH® DM 611 and MOWILITH® LDM 6070 sold by the company Hoechst, and the products RHODAPAS® SD 215 and RHODOPAS® DS 910 sold by the company Rhone-Poulenc; copolymers of styrene, of alkyl methacrylate and of alkyl acrylate; copolymers of styrene and of butadiene; or copolymers of styrene, of butadiene and of vinylpyridine;

polyamides;

vinyllactam homopolymers such as vinylpyrrolidone homopolymers and such as the polyvinylcaprolactam sold under the name LUVISKOL® Plus by the company BASF; and

vinyllactam copolymers such as a poly(vinylpyrrolidone/vinyllactam) copolymer sold under the trade name LUVITEC® VPC 55K65W by the company BASF, poly(vinylpyrrolidone/vinyl acetate) copolymers, such as those sold under the name PVPVA® S630L by the company ISP, LUVISKOL® VA 73, VA 64, VA 55, VA 37 and VA 28 by the company BASF; and poly(vinylpyrrolidone/vinyl acetate/vinyl propionate) terpolymers, for instance the product sold under the name LUVISKOL® VAP 343 by the company BASF.

The alkyl groups of the non-ionic polymers mentioned above for example contain from 1 to 6 carbon atoms.

According to the disclosure, it is also possible to use fixing polymers of grafted silicone type comprising a polysiloxane portion and a portion consisting of a non-silicone organic chain, one of the two portions constituting the main chain of the polymer, and the other being grafted onto said main chain.

These polymers are described, for example, in patent applications EP-A-0 412 704, EP-A-0 412 707, EP-A-0 640 105 and WO 95/00578, EP-A-0 582 152 and WO 93/23009 and U.S. Pat. Nos. 4,693,935, 4,728,571 and 4,972,037.

These polymers may be amphoteric, anionic or non-ionic, and are for example anionic or non-ionic.

Such polymers include, for example, copolymers that can be obtained by free radical polymerization from the monomer mixture formed from:

a) 50 to 90% by weight of tert-butyl acrylate;

b) 0 to 40% by weight of acrylic acid;

c) 5 to 40% by weight of a silicone macromer of formula:

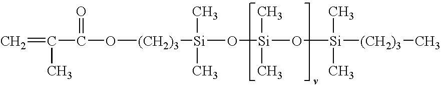

in which v is a number ranging from 5 to 700, the weight percentages being calculated relative to the total weight of the monomers.

Other examples of grafted silicone polymers include polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Another type of silicone fixing polymer that may be mentioned is the product LUVIFLEX® Silk, sold by the company BASF.

Functionalized or non-functionalized, silicone or non-silicone, cationic, non-ionic, anionic or amphoteric polyurethanes or mixtures thereof may also be used as fixing polymers.

For example, the polyurethanes intended by the present invention are those disclosed in patent applications EP 0 751 162, EP 0 637 600, EP 0 648 485 and FR 2 743 297, of which the Applicant is the Proprietor, and patent applications EP 0 656 021 and WO 94/03510 from the company BASF and EP 0 619 111 from the company National Starch.

As polyurethanes that may be suitable for the present disclosure, mention may be made of the products sold under the names LUVISET Pur® and LUVISET® Si Pur by the company BASF.

The concentration of at least one additional fixing polymer which may be used in the compositions according to the present disclosure ranges from 0.1% to 20% by weight relative to the total weight of the composition, such as an amount ranging from 0.5% to 10% by weight.

A person skilled in the art should know how to add the additives without disturbing the properties of the compositions of the disclosure.

For example, the compositions may be in the form of gels. The compositions may have a viscosity greater than 500 cps at a temperature of 25° C. and at a shear rate of 1 s⁻¹.

When the composition according to the disclosure is packaged in an aerosol device, it can comprise at least one propellant, which may be chosen from volatile hydrocarbons, such as N-butane, propane, isobutane, pentane, halogenated hydrocarbons and mixes thereof. It is also possible to use carbon dioxide, nitrous oxide, dimethyl ether (DME), nitrogen or compressed air as the propellant. Mixtures of propellants can also be used. For example, dimethyl ether is used.

For example, the propellant may be present at a concentration ranging from 5 to 90% by weight relative to the total weight of the composition in the aerosol device, such as at a concentration ranging from 10 to 60%.

The composition according to the disclosure may for example be used as a leave-in application on the hair.

Another subject of the disclosure is a method for shaping the hair, comprising the application of a cosmetic composition according to the disclosure. For example, the disclosure relates to a styling method comprising the application of a composition according to the disclosure to the hair, optional rinsing of the hair, then the shaping and drying of the hair.

The examples that follow illustrate the invention without limiting the scope thereof.

### EXAMPLES

The following compositions A and B are produced:

The concentrations are expressed as grams of active material per 100 g of composition.

| | A | B |
|---|---|---|
| Branched sulfonic polyester (1) | 5 | 2 |
| Hydroxypropyl methyl cellulose (2) | 2.2 | — |
| Gellan gum (3) | — | 1 |
| Glycerol | 3 | 1 |
| Propylene glycol | 1 | — |
| Sorbitol | — | 2.7 |
| Sodium chloride | — | 0.25 |
| PEG-40 hydrogenated castor oil (4) | 0.5 | 0.5 |
| Fragrance | 0.2 | 0.2 |
| Preservatives | qs | qs |
| Water | qs 100 | qs 100 |

(1) Eastman AQ 1350
(2) METHOD F 4 M (Dow Chemical)
(3) KELCOGEL F (CP Kelco)
(4) CREMOPHOR CO 40 (Basf)

The compositions exhibit an adequate consistency that allows an easy application and an easy positioning on clean hair. After drying, the hair exhibits a natural hold of long duration.

What is claimed is:

1. A cosmetic composition comprising, in a cosmetically acceptable medium:
at least one branched sulphonic polyester, and
at least one polysaccharide thickener comprising at least one glucose unit.

2. A cosmetic composition according to claim 1, wherein the at least one branched sulphonic polyester is obtained by polycondensation of:
(a) at least one dicarboxylic acid that does not bear a sulphonic functional group,
(b) at least one diol or a mixture of a diol and of a diamine,
(c) at least one monomer comprising two identical or different reactive functional groups chosen from hydroxyl, amino, and carboxyl groups, and at least one sulphonic functional group and
(d) at least one monomer comprising at least three identical or different reactive functional groups chosen from hydroxyl, amino, and carboxyl groups.

3. A cosmetic composition according to claim 2, wherein the at least one branched sulphonic polyester comprises units (e) derived from monomers comprising two different reactive functional groups chosen from hydroxy carboxylic acids and amino carboxylic acids or mixtures thereof.

4. A cosmetic composition according to claim 1, wherein the at least one branched sulphonic polyester is present in an amount ranging from 0.2 to 15% by weight, relative to the total weight of the composition.

5. A cosmetic composition according to claim 1, wherein the at least one branched sulphonic polyester is present in an amount ranging from 0.5 to 10% by weight of relative to the total weight of the composition.

6. A cosmetic composition according to claim 1, wherein the at least one polysaccharide thickener comprising at least one glucose unit is chosen from celluloses, gellan gums, and derivatives thereof.

7. A cosmetic composition according claim 1, wherein the concentration of the at least one polysaccharide thickener comprising at least one glucose unit ranges from 0.05 to 10% by weight relative to the total weight of the composition.

8. A cosmetic composition according claim 1, wherein the concentration of the at least one polysaccharide thickener comprising at least one glucose unit ranges from 0.1 to 5% by weight relative to the total weight of the composition.

9. A cosmetic composition according claim 1, wherein the concentration of the at least one polysaccharide thickener comprising at least one glucose unit ranges from 0.5 to 3% by weight relative to the total weight of the composition.

10. A cosmetic composition according to claim 1, wherein the at least one polysaccharide thickener comprising at least one glucose unit is non-ionic.

11. A cosmetic composition according to claim 1, further comprising at least one additional compound chosen from silicones and non-silicone fatty substances.

12. A cosmetic composition according to claim 11, wherein the non-silicone fatty substances are chosen from mineral, plant, animal and synthetic oils, waxes, fatty esters, ethoxylated and non-ethoxylated fatty alcohols, and fatty acids.

13. A cosmetic composition according claim 1, further comprising at least one adjuvant chosen from thickening agents, co-thickeners, penetrants, fragrances, dyes, plasticizers, buffers, ceramides, pseudoceramides, vitamins or provitamins such as panthenol, opacifiers, reducing agents, emulsifiers, preservatives, mineral fillers, pearlescent agents, flakes, sunscreens, proteins, moisturizers, emollients, demulcents, anti-foaming agents, antiperspirants, free-radical scavengers, bactericides, sequestrants, anti-dandruff agents, antioxidants, basifying agents, and acidifying agents.

14. A cosmetic composition according claim 13, wherein the at least one adjuvant is panthenol.

15. A method for styling or shaping keratin materials comprising applying the cosmetic composition according to claim 1 to the keratin materials.

16. A method according to claim 15, wherein the keratin materials are chosen from human hair.

17. A method for styling or shaping hair comprising:
(a) lying the composition according to claim 1 to the hair;
(b) optional rinsing the hair, and
(c) shaping and drying the hair.
